(19) United States
(12) Patent Application Publication
(10) Pub. No.: US 2009/0149607 A1
(43) Pub. Date: Jun. 11, 2009

(54) SYNTHETIC RECEPTOR

(76) Inventors: Khalku Karim, Cambridgeshire (GB); Sergey Anatoliyovich Piletsky, Bedfordshire (GB); Stuart P. Hendry, Cambridgeshire (GB); Peter Georg Laitenberger, Cambridgeshire (GB)

Correspondence Address: STITES & HARBISON PLLC, 1199 NORTH FAIRFAX STREET, SUITE 900, ALEXANDRIA, VA, 22314, US

(21) Appl. No.: 11/913,497

(22) PCT Filed: Apr. 28, 2006
(86) PCT No.: PCT/GB06/01571

§ 371 Date: Aug. 28, 2008

(30) Foreign Application Priority Data
May 6, 2005 (GB) 0509275.4

(51) Int. Cl. C08F 265/04; G01N 33/00

(52) U.S. Cl. 525/302

(57) ABSTRACT

A polymer capable of selectively binding propofol is prepared from one or more suitable monomers (e.g. N,N-diethylaminoethyl methacrylate) and a cross-linker. It may be a molecularly imprinted polymer. An element (7) of the polymer may be used in a propofol sensor (1) mounted in a confinement structure (3) on a substrate (2).

This invention relates to a synthetic receptor and particularly to a synthetic polymer capable of selectively binding the anaesthetic propofol.

Modern healthcare relies extensively on a range of chemical and biochemical analytical tests on a variety of body fluids to enable diagnosis and management of disease. Medical and technological advances have considerably expanded the scope of diagnostic testing over the past few decades. Moreover, an increasing understanding of the human body, together with the emergence of developing technologies, such as Microsystems and nanotechnology, are expected to have a profound impact on diagnostic technology.

Increasingly diagnostic tests in hospitals are carried out at the point-of-care (PoC), in particular in situations, where a rapid response is a prime consideration and therapeutic decisions have to be made quickly. Despite recent advances in PoC testing, several compelling needs remain unmet. Many of the presently available diagnostic tests rely on the use of sophisticated biological receptors, such as enzymes, antibodies and DNA. Due to their biological derivation, these biomolecules typically suffer from a number of limitations when used in sensing applications, for example, poor reproducibility, instability during manufacture, sensitivity to environmental factors, such as pH, ionic strength, temperature etc., and problems associated with the sterilisation process.

A promising route to overcome these issues is offered by synthetic polymer-based receptors, such as molecularly imprinted polymers (MIPs). Synthetic receptors avoid many of the disadvantages associated with biological receptors. Molecular imprinting, for example, is a generic and cost-effective technique for preparing synthetic receptors, which combine high affinity and high specificity with robustness and low manufacturing costs. In addition, MIP receptor materials have already been demonstrated for a wide range of clinically relevant compounds and diagnostic markers. In contrast to biological receptors, synthetic receptors, and particularly MIPs, typically are stable at low and high pH, pressure and temperature, are inexpensive and easy to prepare, tolerate organic solvents, may be prepared for practically any analyte, and are fully compatible with micromachining and microfabrication technology.

Molecular imprinting may be defined as the process of template-induced formation of specific recognition sites (binding or catalytic) in a material, where the template directs the positioning and orientation of the material's structural components by a self-assembling mechanism. The material itself could be oligomeric, polymeric (for example, organic MIPs and inorganic imprinted silica gels) or two-dimensional surface assemblies (grafted monolayers).

In many applications, for example, where the receptor is to be used repeatedly without significant regeneration between applications, the use of so-called non-covalent MIPs is generally preferred, in particular in sensing applications. As the template/analyte is only weakly bound by non-covalent interactions to these receptor materials, it can be relatively easily removed from the synthetic receptor and the sensor regenerated for a new measurement. In general, non-covalent imprinting is easier to achieve and applicable to a wider spectrum of templates.

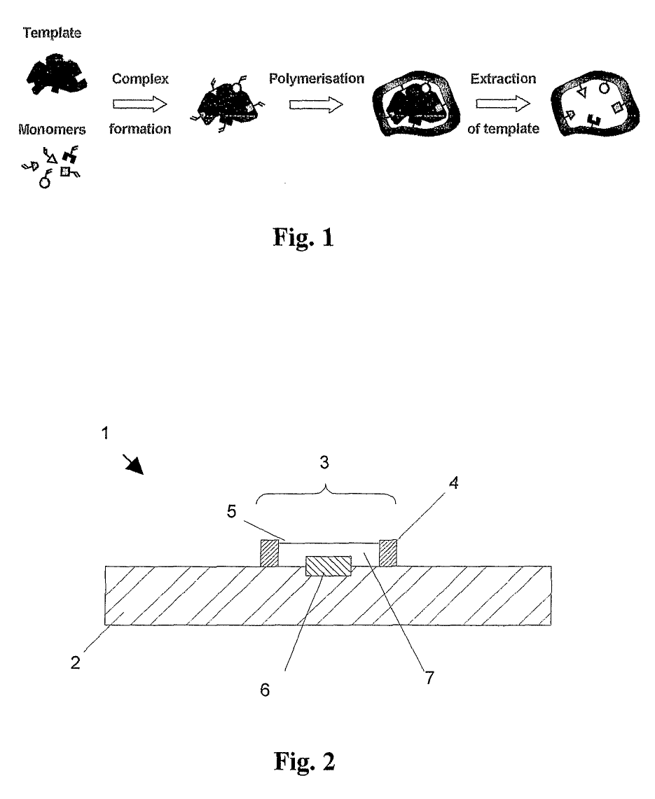

In non-covalent MIPs, the monomer(s) contained within the polymer interact with the template through non-covalent interactions, for example, hydrogen bonding, electrostatic interaction, coordination-bond formation etc. FIG. 1 shows a schematic representation of the self-assembly of a MIP from monomeric starting materials to form a polymer having binding sites with specificity for the template, i.e. the target analyte or a structural analogue thereof, and the subsequent elution or extraction of the template.

This technique has been employed to create successfully MIPs for a range of chemical compounds, ranging from small molecules (up to 1200 Da), such as small organic molecules (e.g. glucose) and drugs, to large proteins and cells. The resulting polymers are robust, inexpensive and, in many cases, possess affinity and specificity that is suitable for diagnostic applications. The high specificity and stability of MIPs render them promising alternatives to enzymes, antibodies, and natural receptors for use in sensor technology.

For example, WO 02/00737 discloses a system for the detection of the intravenous anaesthetic propofol. In particular, the synthesis of a non-covalent MIP capable of binding propofol is described. This MIP is composed of methacrylic acid (MAA) as the monomer and ethylenedimethylacrylic acid (EDMA) as the cross-linker. The document also discusses a method for measuring the propofol concentration in a blood sample, which involves the extraction of propofol from the blood sample using methanol and the adsorption of propofol from the extract on the MIP. After adsorption on the MIP, the propofol is then extracted from the polymer and the propofol concentration is determined using HPLC or optical techniques. However, the methods disclosed tend to suffer from a number of disadvantages, including being off-line, tending to be cumbersome to carry out, requiring the use of methanol for the extraction of propofol from a blood sample and of additional chemicals for the analysis process and being generally slow to use.

A number of methodologies have been proposed to introduce synthetic polymer-based receptors, including MIPs, into devices for the analysis of clinically relevant analytes, but to date they have only had limited success. One of the main limitations associated with the development of MIP assays and sensors has been the absence of a general procedure for MIP preparation. Traditionally, the choice of polymer composition is based on information available from the literature concerning the behaviour of similar systems, the individual experience of chemists, and extensive experimental trials and is therefore often sub-optimal. The polymer compositions identified are typically synthesised and tested in the laboratory with respect to their properties, e.g. binding affinity for the template and other compounds, which may be present in the sample. Based on the experimental results, the polymer composition can be further refined to yield synthetic receptors with suitable binding properties for the application in hand.

A more advanced protocol for the design of MIPs involves a combinatorial method, whereby the best composition is selected on the basis of simultaneous synthesis and testing of tens to hundreds of imprinted polymers prepared on the small scale.

Properties which may be optimised as part of the procedure include, but are not limited to, binding affinity, capacity, speed of response, regeneration, cross-sensitivity to other analytes and/or operation in real samples, solvents or media, such as water or blood.

However, there remains in the art a need for materials capable of selectively binding propofol.

Accordingly, the present invention provides a sensor comprising a polymer for binding propofol composed of a monomer selected from at least one or more of N,N-diethylamino ethyl methacrylate (DEAEM), acrylamide, 2-(trifluoromethyl)acrylic acid (TFMAA), itaconic acid and ethylene glycol methacrylate phosphate (EGMP), and a cross-linker.

The present invention also provides the use of the above-defined polymer for binding propofol and a molecularly imprinted polymer imprinted with propofol (i.e. synthesised in the presence of propofol) having the above-defined components.

The present invention will now be described with reference to the accompanying drawings, in which:

FIG. 1 shows a schematic representation of the fabrication process for a MIP; and

FIG. 2 shows structure of a sensor incorporating a synthetic receptor.

The present invention relates to the composition of polymers for the detection of the clinically relevant target analyte, propofol. In particular, it relates to polymer compositions, which were optimised for use in real samples, i.e. blood or physiological solutions, high binding affinity, high binding speed and simple regeneration without the need for significant sample preparation. These materials can be prepared in the form of molecularly imprinted polymers (MIP) and non-imprinted polymers (NIP).

There are a large number of potential monomers, which may be used in the synthesis of MIPs or NIPs, such as acrylates, amides, vinyl and allyl monomers, urethanes, phenols, boronates, organosiloxanes, carbonate esters, sulfonic acids, etc. See for example, M. Komiyama et al. Molecular Imprinting: From Fundamentals to Applications, Wiley-VCH Verlag GmbH & Co KGaA, Weinheim (2003), G. Wulff Angew. Chem. Int. Ed. Engl. 34, 1812 (1995), and S. Subrahmanyam et al. Biosensors & Bioelectronics 16, 631 (2001). The polymer compositions described herein were identified and optimised by a careful study of the properties of these monomers with respect to the binding to the analyte and potential interferents and other relevant substances using theoretical and experimental methodologies. Similarly, the properties of the polymer can be analysed as a function of the solvent or medium in which the analysis or interaction takes place. For medical applications, where the samples predominantly exist as blood samples, urine samples, dialysates, saliva samples, etc., it is often preferred to carry out the sample analysis or diagnostic test directly in these samples. Hence, it is preferable for the MIP or NIP to be optimised for operation in aqueous media, typically under physiological conditions.

Based on the results of the present analysis a number of monomers were identified as suitable candidates for the synthesis of synthetic receptors for propofol. The present invention provides synthetic receptors prepared with a monomer selected from at least one or more of: N,N-diethylamino ethyl methacrylate (DEAEM), acrylamide, 2-(trifluoromethyl)acrylic acid (TFMAA), itaconic acid and ethylene glycol methacrylate phosphate (EGMP).

By synthetic receptor is meant a synthetic polymer which is capable of selectively binding a specific analyte.

Each of these monomers, or a mixture thereof, can be used together with a cross-linker to prepare synthetic receptors for propofol. The synthetic receptors can be prepared in the form of imprinted polymers or non-imprinted polymers. Examples of protocols for the synthesis of these materials are described hereinbelow.

In addition, in order to tailor the properties of the polymer it may also be desirable to incorporate other monomers into the polymer. For example, it may be desirable to construct a polymer which has a good binding affinity to the analyte to be bound, but which can also be regenerated in a straight-forward manner. That is, a polymer which can selectively bind propofol but from which the propofol may subsequently be removed to allow re-use of the polymer. Furthermore, it may be desirable to consider other polymer properties, such as mechanical stability, binding or sensitivity to other compounds, characteristics of operating in a particular environment (e.g. solvent used), integration of the polymer with a support or with a sensor, biocompatibility of the surface, etc. in addition to the binding affinity for the analyte of interest and the ease of regeneration, in order to synthesise a polymer, which is optimised for a particular application. This objective can be achieved by using a mixture of monomers containing a monomer with high binding affinity as identified herein, and another monomer with low binding affinity, in a suitable ratio. Alternatively, the different monomers in the mixture may cooperate with each other in order to provide the desired effects, e.g. they may provide additional binding at different sites or places around the analyte or molecule to be bound. This effect can, for example, be used to increase the binding of the molecule to the polymer or to improve the cross-sensitivity in binding to other substances which may be contained in the sample. Furthermore, adding additional monomers to the mixture may also change the biocompatibility of the surface. Employing polymer mixtures in this manner, enables the properties of the polymer to be tailored to the particular requirement of the application.

The functional monomer (i.e. N,N-diethylamino ethyl methacrylate (DEAEM), acrylamide, 2-(trifluoromethyl)acrylic acid (TFMAA), itaconic acid and/or ethylene glycol methacrylate phosphate (EGMP)) is preferably present at a minimum of 5 mol %, more preferably 10 mol % and most preferably 20 mol %, and a maximum of 100 mol %, more preferably 95 mol % and most preferably 90 mol %, based on the total monomer content.

While the synthesis of MIPs and NIPs disclosed in this document has been carried out using EGDMA as cross-linker, other cross-linkers can be used to prepare suitable synthetic receptors.

The cross-linker may be included to fix the template-binding sites firmly in the desired structure as well as to influence the porosity of the MIP or NIP. The cross-linker must be capable of reacting with the monomers to cross link the polymer and the cross-linker should preferably be of similar reactivity to the monomer. Suitable cross-linkers include, but are not limited to, ethylene glycol dimethacrylate (EDMA), glycerol dimethacrylate (GDMA), trimethylacrylate (TRIM), divinylbenzene (DVB), methylenebisacrylamide and piperazinebisacrylamide, phenylene diamine, dibromobutane, epichlorohydrine, trimethylolpropane trimethacrylate and N,N′-methylenebisacrylamide. The mole ratio of monomer to cross-linker is preferably from 1:1 to 1:15. Mixtures of monomers and cross-linkers may also be used.

Polymers made in this manner showed superior performance, for example in terms of their binding affinity for propofol, than the imprinted polymer based on methacrylic acid (MAA) as the monomer, described in WO 02/00737.

Optimal monomer-template ratios to be used for the polymer composition and synthesis have also been identified. Preferred ratios derived from the present analysis for two particular polymers are shown in Table 1.

TABLE 1
Composition of MIPs synthesised.

| | | | Template:Monomer |
|---|---|---|---|
| Polymer | Monomer | Template | Ratio (moles) |
| M1 | DEAEM | Propofol | 1:4 |
| M2 | Acrylamide | Propofol | 1:4 |

In addition simply to analysing the binding affinity of the monomer-template complexes, the analysis of the polymer properties can be extended to other polymer parameters and can be used to optimise other polymer properties. For example, one can screen the monomers identified against analytes, which may be present in the sample and which may act as interferents to the planned measurement or process. One can therefore select monomers, which bind strongly with the target analyte, i.e. propofol, but bind or interact weakly, if at all, with other substances present in the sample, e.g. morphine, glucose or albumin.

In the case of propofol being the target analyte, we found, for example, that EGMP strongly binds to propofol and alfentanil. A polymer containing EGMP as a monomer would therefore be able to act as a synthetic receptor for both propofol and alfentanil. In contrast, DEAEM interacts strongly with propofol and only weakly with alfentanil. A polymer containing DEAEM as the monomer will therefore interact strongly with propofol, while it will show only little or no cross-sensitivity to alfentanil. This DEAEM-containing polymer would therefore be able to discriminate between propofol and alfentanil in a solution containing both analytes.

Although the MIP is preferably synthesised in the presence of propofol, it may also be synthesised in the presence of an analogue of propofol. The analogue must be sufficiently stereoelectronically similar to propofol to render the MIP capable of binding propofol itself.

Preferred embodiments of the present invention relate to the creation of non-imprinted polymers (NIP) as receptor materials for propofol. In particular, these NIPs are composed of either DEAEM, EGMP or acrylamide or a mixture thereof as monomers and EGDMA as cross-linker and are synthesised, for example, using the procedures outlined herein.

The monomers identified above bind strongly to the target analyte of interest, i.e. propofol. Moreover, the interaction of these monomers with other analytes, which may be present in the solution can also be evaluated using a similar approach. It is therefore possible to select monomers, which interact strongly with the target analyte, for example, propofol, but weakly with other analytes in the sample being tested. These monomers, together with a suitably chosen cross-linker, can therefore be used to synthesise non-imprinted polymers (NIPs), which act as the synthetic receptors for the analyte of interest. These polymers showed high binding affinity for propofol and low binding for a number of analytes, which may be present in a sample, such as albumin and morphine.

The non-imprinted polymer has the same composition and synthesis procedure as the corresponding MIP, except that the target analyte or template is not present in the mixture during the polymerisation. A subsequent washing step to remove the template from the polymer (either partially or fully) is therefore also not required. Hence, the synthesis of NIPs is generally less complex and costly in comparison to the corresponding molecularly imprinted polymer (MIP).

The NIPs utilise the monomers listed above and may be synthesised from the following composition:

5 g DMF (dimethylformamide)
1 g monomer
4 g cross-linker (EGDMA)
0.1 g initiator (1,1′-azobis (carbonitrile))

The polymers were synthesised as non-imprinted polymers (NIPs), i.e. in the absence of the template during the polymerisation process. The chemicals were mixed together and the polymerisation was carried out by UV for 20 min using a Hönle 100 UV lamp (intensity 0.157 W/cm²). The mixture was then kept at 80° C. for one day. Other variations in the composition and different synthetic routes can be made and are known to those skilled in the art.

In principle, all the monomers identified in the study can be used together with a suitable cross-linker, such as EGDMA to prepare synthetic receptors, in particular for NIPs to bind propofol in aqueous systems or samples, such as those typically used in clinical diagnostic applications.

In order to characterise the NIPs with respect to their binding properties, following the polymerisation, the polymers were ground and sieved in methanol. The fraction between 25 μm and 106 μm was collected. 10 mg of each polymer were packed in 1 ml solid phase extraction cartridges and the binding of propofol to each polymer was evaluated by measuring the binding capacity of each column.

The binding properties of the polymers were tested with a propofol concentration of 133 μg/ml in water. The cartridges were conditioned with 2 ml water prior to binding. Solutions containing propofol (in 2 ml or 4 ml aliquots) were passed through each cartridge until saturation of the cartridge was observed when a 50% breakthrough of the loaded concentration was observed by UV (ultraviolet) spectrophotometry (at a wavelength of 272 nm).

The monomers identified in the present study show high binding capacity and therefore strong binding affinity to the relevant target analyte. These monomers are therefore well suited for use in the fabrication of synthetic receptors, such as MIPs or NIPs for propofol.

Table 2 shows the binding capacity of each polymer as a percentage of the initial polymer weight. For example, for the acrylamide polymer, 1.3 mg of propofol bound to the 10 mg cartridge when 50% breakthrough was observed, giving a binding capacity of 13% (from 133 μg/ml solution in water).

TABLE 2
Binding capacity of the different polymers identified.

| Monomer | Binding Capacity to Propofol |
|---|---|
| DEAEM | 23% |
| EGMP | 18.5% |
| Acrylamide | 13% |
| TFMAA | 10.8% |
| Itaconic acid | 10.0% |
| Methacrylic acid | 6.7% |
| Vinylimidazole | 2.6% |

Polymers made with just the cross-linker (i.e. in the absence of any monomer) have shown no binding to the templates under investigation. The observed absence of any binding of the template to the polymer made from the cross-linker (EGDMA) indicates that the cross-linker will not interfere with the monomer-template complex.

The monomers identified herein, namely DEAEM, acrylamide, itaconic acid, EGMP, 2-(trifluoromethy)acrylic acid, show high binding capacity and therefore strong binding affinity to the relevant target analyte. These monomers are therefore well suited for use in the fabrication of synthetic receptors, such as NIPs and MIPs for propofol. In particular, the polymers identified for propofol showed stronger binding affinity to propofol than the polymer based on MAA as the monomer, which have been previously suggested (see WO 02/00727). In order to create synthetic receptors with strong binding affinity for propofol, the use of DEAEM, acrylamide, itaconic acid, EGMP, 2-(trifluoromethy)acrylic acid or a mixture thereof as monomers in a MIP or NIP for use as receptors in aqueous systems is therefore preferable to methacrylic acid.

Rather than using just one monomer in the polymer synthesis, it can be advantageous to use a mixture of several monomers in a particular synthetic receptor in order to optimise the properties of the receptor for a particular analyte or sample, e.g. based on the pH of the sample, the solvent or sample medium used or the presence of other species interfering with the measurement.

Further embodiments of the present invention relate to the creation of imprinted polymers.

In principle, all the monomers identified in this document can be used together with a suitable cross-linker, such as EGDMA, to prepare synthetic receptors, in particular MIPs, to bind propofol in aqueous systems or samples, such as those typically used in clinical diagnostic applications.

The general principle for the synthesis of the polymers, which are examples of preferred embodiments of the invention, is described below and examples of the monomer to template ratios used during the synthesis are detailed in Table 1.

The monomer and template concentration is 20% of the total weight of the reactants and the cross-linker EGDMA is the other 80% for the polymers synthesised. The same quantity of solvent (DMF) was added by weight with respect to the reaction mixture. 1% of radical initiator (azobisisobutyronitrile, AIBN) was added with respect to the total monomer:template:cross-linker composition by weight.

The polymers M1 and M2 were imprinted with propofol as the template. The polymerisation was carried out at 80° C. for one day except for M1, which was polymerised by UV for 60 mins using a Hönle 100 UV lamp (intensity 0.157 W/cm²) and then kept at 80° C. for one day.

The template has been extracted by extensive washing with methanol. It is also possible to remove the template by other means known to those skilled in art, such as by electrodialysis.

The polymers were ground and sieved in methanol. The fraction between 25 μm and 106 μm was collected and 10 mg of the polymer were then packed in SPE cartridges columns. The polymers were then analysed using a UV spectrophotometer to ensure that there is no template leeching prior to the binding experiments. This was carried out by measuring the absorbance of the washings from methanol, water and phosphate-buffered saline, PBS (aqueous solution of 140 mM NaCl, 3 mM KCl and 10 mM phosphate buffer at pH 7.4), to ensure the absorbance and the wavelength of detection for the template were at baseline levels prior to commencement of the binding experiments.

Binding experiments were carried out in PBS at physiological concentrations, spiked with 12.5 mg/ml propofol. The cartridges were conditioned with 2 ml PBS prior to binding. Propofol solutions (volumes of 2 ml) were passed through each cartridge and binding was observed by monitoring the UV adsorption in the initial solution and the eluent at the appropriate wavelengths. The UV absorbance of the 2 ml aliquots was compared before and after loading onto the SPE cartridges to calculate the percentage bound to the polymer with respect to the original concentration. Cross-reactivities to other substances were measured in a similar fashion. Table 3 summarises the results of the binding experiments.

TABLE 3
Result of binding experiments of MIPs.

| Polymer | Template imprinted | Template (% bound) |
|---|---|---|
| M1 | Propofol | 72 |
| M2 | Propofol | 55 |

M1 shows the highest binding for propofol. Furthermore, the polymer showed no or only low cross-reactivity to alfentanil, morphine and albumin. Similarly, polymer M2 also showed a high binding affinity for propofol, albeit weaker than M1, with low or no cross-reactivity for morphine and albumin. The cross reactivity of M2 to alfentanil is slightly higher than M1, but still low (<25% of propofol bound at an propofol concentration of 17 μl/ml).

Table 4 shows that as the concentration of alfentanil in the solution is lowered the cross-reactivity of MIPs M1 and M2 to alfentanil drops significantly. This result suggests that the selectivity of the propofol-imprinted polymer can be enhanced by changes in the polymer composition. With decreasing alfentanil concentration, the quantity of non-specifically bound material decreases significantly. The same effect can be achieved for a given concentration of the interfering species in the solution by varying the composition of the polymer or the amount of accessible polymer.

TABLE 4
Cross-reactivity of MIP M1 and M2 imprinted with propofol
to alfentanil at different propofol concentrations.

| | Concentration | M1 | M2 |
|---|---|---|---|
| Interfering analyte | (μg/ml) | (% bound) | (% bound) |
| Alfentanil | 50 | 28 | — |
| Alfentanil | 21 | 15 | 63 |
| Alfentanil | 17 | 7 | 25 |

The performance of the polymers M1 and M2 were also compared with a non-covalent MIP synthesised using the protocol previously disclosed in WO 02/00737. This procedure uses methacrylic acid as the monomer and hexane as a porogen. This polymer, M1 and M2 were tested under the same conditions (using a 10 mg SPE cartridge at a concentration of 12.5 μg/ml of propofol in PBS).

The MIP disclosed in WO 02/00737 bound 50% of the propofol present in the solution prior to loading onto the SPE cartridge, while polymer M1 bound 72% of the propofol present. M2 bound 55% of the propofol present in the solution prior to loading onto the column. These results demonstrate that the polymers M1 and M2 made for propofol outperform that disclosed in WO 02/00737. It was also observed that the mechanical stability of the polymer disclosed in WO 02/00737 was very weak, which is expected to lead to problems when integrating the polymer onto a sensor platform. In contrast, polymers M1 and M2 were far more rigid and mechanically stable. In addition, the speed of binding of the analyte to the MIPs M1 and M2 was very high (less than 1 min).

The synthetic receptors disclosed in this document can be used in a variety of applications. In particular, the devices incorporating and methods and applications of using these novel receptors as sorbents in separation and chromatography columns or as receptor materials in sensors are subjects of the invention. One preferred embodiment of the invention relates to a sensor for the measurement of the concentration of propofol in a fluid sample, which is constructed by the deposition of a synthetic material synthesised according to the methods outlined above on a transducer element.

In another embodiment of the invention, the synthetic receptors are used as sorbents for solid-phase extraction or filtration. Furthermore, they can also be employed as sorbents in HPLC columns. For these purposes, the MIPs or NIPs are typically formed as a plastic, then ground into smaller particles, sieved to select the desired particle size and packed into columns. The MIPs or NIPs can also be prepared in the form of microspheres or membranes. Furthermore, they can be attached to membranes or other supports.

The synthetic receptors can also be employed in chemical sensors. In one embodiment, the synthetic receptor is used as an adsorption medium to extract the analyte to be detected from a sample or an extract thereof. The analyte is then desorbed from the synthetic receptor in a further extraction step and is detected. A typical example of this approach is described in WO 02/00737 and the MIPs and NIPs of the present invention may be applied in this manner.

The synthetic receptors disclosed herein, either in the form of an imprinted polymer or in the form of a non-imprinted polymer, can also be directly integrated with transducers for the detection, concentration measurement or monitoring of one or more target analytes in a sample. Examples of this approach are given, for example, in GB 2 337 332. Other integration approaches are known to those skilled in the art. In these embodiments, the synthetic receptor(s) for the target analyte(s), either in the form of a MIP or a NIP, is localised in close proximity to the transducer element. Upon contact with the sample, the target analyte, if present in the sample, (to some extent) interacts with and/or binds to the receptor. This interaction or binding is detected by the transducer and transformed into a measurable signal, e.g. an electrical or optical signal. A wide range of transduction techniques are known, including electrochemical (e.g. amperometric, conductometric or potentiometric, in particular ISFETs (ion-sensitive field effect transistors)), optical (e.g. fluorescence, luminescence, adsorption, spectrometric, etc.), gravimetric, resonant, magnetic, thermal, surface-acoustic wave, strain, position or displacement or time-of-flight techniques, to name but a few.

The imprinted or non-imprinted polymers described herein may also be integrated with a micromachined sensor in order to construct a device for the detection, concentration measurement or monitoring of an analyte of interest. The sensor may use any of the transduction principles mentioned above. In order to reduce the size or to increase the robustness of the sensor, it is advantageous to include means to localise the polymer in the vicinity of the transducer and to enhance its adhesion to the surface of the transducer or substrate in the sensor construction. Accordingly, this application also provides a sensor, which comprises a (typically planar) substrate, a confinement structure disposed on the substrate, wherein the confinement structure comprises at least a first limiting structure defining a first interior space, a transducer proximal to the first interior space, and a synthetic polymer capable of selectively binding a first analyte, within the confinement structure, wherein the synthetic polymer is a polymer as described herein. Examples of such confinement structures and details of the standard techniques for their fabrication are disclosed, for example, in U.S. Pat. No. 5,376,255 and U.S. Pat. No. 6,440,296.

A possible structure of the sensor is shown in FIG. 2. The reference numerals are: the sensor 1, the substrate 2, the confinement structure 3, a first limiting structure 4, a first interior space 5, a transducer 6 and a synthetic receptor 7, preferably in the form of imprinted or non-imprinted polymers disclosed in this document.

As well as the first limiting structure the confinement structure may further comprise a second limiting structure defining a second interior space, the second interior space containing the first interior space. The confinement structure may also further comprise one or more further limiting structures defining one or more further interior spaces, the one or more further interior spaces each containing a preceding interior space. The confinement structure and hence the first, second and further limiting structures may be any shape but are preferably annular.

In addition, the sensor may also comprise additional transducer elements and/or confinement structures, which contain polymers capable of selectively binding further analytes, other receptor materials (e.g. enzymes, antibodies, etc.) or reference materials.

The present invention also provides a method of detecting a target species in a sample comprising a sensor as defined hereinabove with a sample containing or suspected to contain the target species.

In order to facilitate the immobilisation of a synthetic receptor on a support, the support can be modified. For example, the surface of the support/transducer may be modified with agents enhancing the polymer adhesion by the attachment of silanes or thiols containing double bonds. These groups can then react with the constituents of the synthetic receptor either before or after polymerisation to provide a chemical link between the receptor and the transducer.

Immobilisation of adequate functional groups or free radical initiators onto the surface of the sensor may be realised by linking molecules which attach to the surface of the substrate. The covalent attachment of the MIP or NIP to the substrate is then performed via coupling reactions between the chemically modified surface and the MIP or NIP.

Immobilisation may be achieved on a variety of materials, such as silicon, silicon oxide, silicon nitride and metals, using a wide range of chemistries. See for example Bartlett P N Modification of sensor surfaces, Handbook of chemical and biological surfaces, Edited by Taylor R F and Schultz J S, Institute of Physics Publishing (1996). Examples of two convenient routes use a silane or thiol. Further polymerisation of the MIP at this level ensures the stable and robust preparation of the sensor.

In order to improve the speed of response or sensitivity of the sensor, polymeric porogens, such as polyvinyl acetate and polyethylene glycol, can be added to the polymerisation mixture prior to polymerisation of the polymer. See, for example, Sergeyeva T. A., et al. (2003). Macromolecules, 36, 7352-7357 and Schmidt R. H. et al. (2004). Advanced Materials, 16, 719-722.

1. A sensor comprising a polymer for binding propofol composed of a monomer selected from at least one or more of N,N-diethylamino ethyl methacrylate (DEAEM), acrylamide, 2-(trifluoromethyl)acrylic acid (TFMAA), itaconic acid and ethylene glycol methacrylate phosphate (EGMP), and a cross-linker.

2. A sensor as claimed in claim 1, wherein the cross-linker is selected from ethylene glycol dimethacrylate (EDMA), glycerol dimethacrylate (GDMA), trimethylacrylate (TRIM), divinylbenzene (DVB), methylenebisacrylamide and piperazinebisacrylamide (which are particularly suitable for cross linking acylamides), phenylene diamine, dibromobutane, epichlorohydrine, trimethylolpropane trimethacrylate and N,N′-methylenebisacrylamide.

3. A sensor as claimed in claim 1, wherein the mole ratio of monomer to cross-linker is from 1:1 to 1:15.

4. A sensor as claimed in claim 1, wherein the polymer is a molecularly imprinted polymer capable of binding propofol.

5. A sensor as claimed in claim 4, wherein the polymer is a molecularly imprinted polymer imprinted with propofol.

6. A sensor as claimed in claim 1, wherein the sensor further comprises a substrate, and a confinement structure disposed on the substrate, wherein the confinement structure comprises at least a first limiting structure defining a first interior space for containing the polymer; and a transducer proximal to the first interior space.

7. A method of binding propofol comprising contacting a solution containing it with a polymer composed of a monomer selected from N,N-diethylamino ethyl methacrylate (DEAEM), acrylamide, 2-(trifluoromethyl)acrylic acid (TFMAA), itaconic acid, ethylene glycol methacrylate phosphate (EGMP), and mixtures thereof, and a cross-linker for binding propofol.

8. A method as claimed in claim 7, wherein the cross-linker is selected from ethylene glycol dimethacrylate (EDMA), glycerol dimethacrylate (GDMA), trimethylacrylate (TRIM), divinylbenzene (DVB), methylenebisacrylamide and piperazinebisacrylamide (which are particularly suitable for cross linking acylamides), phenylene diamine, dibromobutane, epichlorohydrine, trimethylolpropane trimethacrylate and N,N′-methylenebisacrylamide.

9. A method as claimed in claim 7, wherein the mole ratio of monomer to cross-linker is from 1:1 to 1:15.

10. A method as claimed in claim 7, wherein the polymer is a molecularly imprinted polymer imprinted with propofol.

11. A molecularly imprinted polymer imprinted with propofol composed of a monomer selected from N,N-diethylamino ethyl methacrylate (DEAEM), acrylamide, 2-(trifluoromethyl)acrylic acid (TFMAA), itaconic acid, ethylene glycol methacrylate phosphate (EGMP), and mixtures thereof, and a cross-linker.

12. A molecularly imprinted polymer as claimed in claim 11, wherein the cross-linker is selected from ethylene glycol dimethacrylate (EDMA), glycerol dimethacrylate (GDMA), trimethylacrylate (TRIM), divinylbenzene (DVB), methylenebisacrylamide and piperazinebisacrylamide (which are particularly suitable for cross linking acylamides), phenylene diamine, dibromobutane, epichlorohydrine, trimethylolpropane trimethacrylate and N,N′-methylenebisacrylamide.

13. A molecularly imprinted polymer as claimed in claim 11, wherein the mole ratio of monomer to cross-linker is from 1:1 to 1:15.
